(19) United States
(12) Patent Application Publication
(10) Pub. No.: US 2019/0038205 A1
(43) Pub. Date: Feb. 7, 2019

(54) DYNAMOMETER

(71) Applicant: Steven A. Gold, Tiburon, CA (US)

(72) Inventor: Steven A. Gold, Tiburon, CA (US)

(21) Appl. No.: 16/054,998

(22) Filed: Aug. 3, 2018

Related U.S. Application Data
(60) Provisional application No. 62/540,995, filed on Aug. 3, 2017.

(51) Int. Cl. A61B 5/22; A61B 5/00; A61B 5/11; G01L 1/24; G01L 5/22

(52) U.S. Cl. CPC A61B 5/225; A61B 5/6826; A61B 5/1125; A63B 2220/56; G01L 5/226; A61B 2503/10; G01L 1/247

(57) ABSTRACT

A dynamometer may include a sensor embedded within and distributed uniformly within a pad. The pad may be configured for gripping by a human hand or foot such that grip force of particular elements of the human hand or foot may be sensed by the sensor.

### FIELD OF THE INVENTION

The present disclosure relates generally to a dynamometer and particularly to a dynamometer that may detect and indicate pressure or force applied by individual portions of the hand or foot such as fingers.

### BACKGROUND

Handgrip dynamometers are instruments for measuring the maximum isometric contraction strength of the hand and forearm muscles. Handgrip dynamometers are used for testing handgrip strength of patients suffering from conditions that impair hand strength and for tracking improvements during rehabilitation. They are also used by athletes involved in strength training or participating in sports in which the hands are used for catching, throwing or lifting such as gymnasts, tennis players and rock climbers.

Prior art handgrip dynamometers, however, are limited in that they may only measure strength of the whole hand. Prior art handgrip dynamometers are also limited in that they measure only contraction strength. In many cases, whole hand or contraction strength measurements are not adequate.

### SUMMARY OF THE INVENTION

The dynamometers disclosed herein may measure strength at a more granular level by measuring and providing feedback regarding pressure applied by individual portions of the hand or foot, not just the hand as a whole. For example, in one embodiment, a dynamometer may display a representation of pressure applied by individual fingers. In another embodiment, a dynamometer may display a representation of force applied by individual fingers during expansion or spreading of the hand. These embodiments represent improvements over the limitations of the prior art and may help patients and athletes by providing additional measures of conditioning or improvements.

### BRIEF DESCRIPTION OF THE DRAWINGS

FIG. 1 illustrates a perspective view of an exemplary dynamometer and corresponding prompting device.

FIG. 2 illustrates a perspective view of the exemplary dynamometer of FIG. 1 and the corresponding prompting device.

FIG. 3A illustrates a perspective view of an exemplary dynamometer.

FIG. 3B illustrates a perspective view of another exemplary dynamometer.

FIG. 4A illustrates a perspective of another exemplary dynamometer.

FIG. 4B illustrates a magnified view of the exemplary dynamometer of FIG. 4A.

FIG. 5 illustrates a block diagram of an exemplary dynamometer.

### DETAILED DESCRIPTION

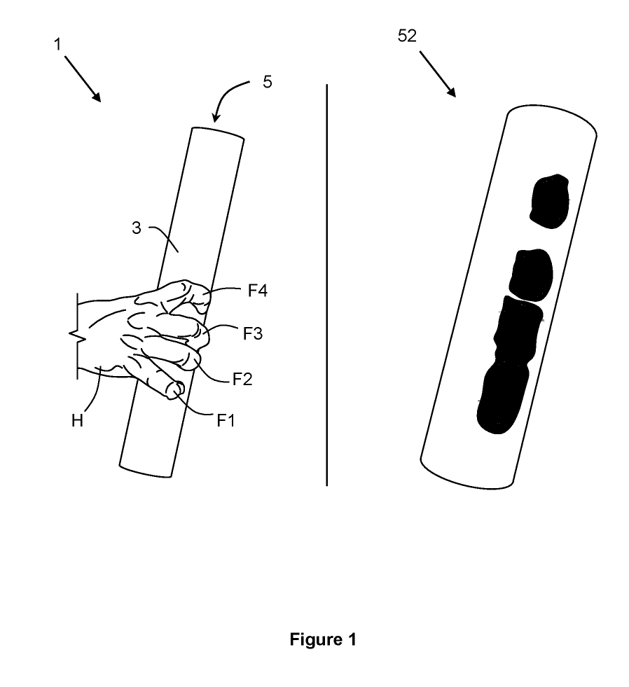

FIG. 1 illustrates on the left a perspective view of an exemplary dynamometer 1. The dynamometer 1 may include a pad 3 that has a distributed sensor 3. The pad 3 is wrapped around to resemble a cylinder 5. Although for purposes of illustration, the pad 3 is disclosed herein as wrapped around to resemble a cylinder, the pad 3 is not so limited and may be wrapped to resemble any shape (e.g., prism, cone, sphere, oval, etc.) that a human hand may grip. Also, more than one piece of pad 3 may be disposed (e.g., on opposite or substantially opposite sides of a cylinder, prism, cone, sphere, oval, disc, etc.) for gripping by a human hand such that grip strength of particular elements of the human hand may be sensed by the sensor or plurality of sensors distributed within the pad or fabric.

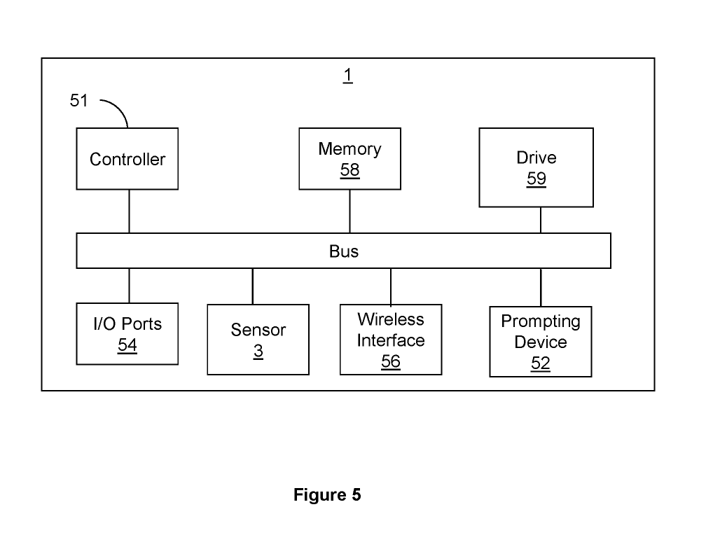

As shown in FIG. 5, the dynamometer 1 may also include a controller 51 and a prompting device 52. FIG. 1 illustrates on the right the prompting device 52, in this case a display on a screen of a computer monitor that displays the sensed compression or contraction force the person's hand H applies to the cylinder 5. The display is in the form of a “weather map” in which darker colors indicate higher force while lighter colors indicate lower force. Notice that the illustration shows that, in this example, almost all of the force is being applied by the tip of the fingers F1-F4.

The controller 51 may reside within the cylinder 5 of the dynamometer 1 or external to the cylinder 5. The distributed sensor 3 senses compression or contraction force applied by the hand H or fingers F1-F4. The controller 51 is operatively coupled to the distributed sensor 3 and the prompting device 52 and controls the distributed sensor 3 and prompting device 52 when the dynamometer 1 is in use. The dynamometer 1 may, for example, include a connector or I/O port 54 (e.g., USB or micro USB) to connect the dynamometer 1 to an external controller 51 or prompting device 52 or the dynamometer 1 may include a wireless (e.g., Bluetooth) interface 56 to connect the dynamometer 1 to the external controller 51 or the prompting device 52.

The distributed sensor 3 is uniformly distributed on the dynamometer 1. The pad in which the distributed sensor 3 is embedded may have at least 91 cm² surface area to accommodate a person's hand and the distributed sensor 3 may be configured to sense compression force applied throughout the surface area. The pad 3 may, for example, correspond to fabric produced by BeBop Sensors of Berkeley, Calif. The distributed sensor 3 senses the compression force throughout the surface area, including sensing if more pressure is applied to one area vs another as shown in the “weather map” on the right side of FIG. 1. The distributed sensor 3 may operate in accordance with the operational details disclosed in U.S. Pat. No. 9,076,419 hereby incorporated by reference in its entirety. Upon sensing the compression force applied, the distributed sensor 3 transmits a signal including the sensed compression force to the controller 51.

The controller 51 (e.g., a microprocessor, a central processing unit of a computing device, or an integrated chip) is operatively connected to the distributed sensor 3 and the prompting device 52. The controller 51 receives the sensed compression force from the distributed sensor 3. The controller 51, in turn, calculates and produces a signal to be sent to the prompting device 52 for displaying the feedback. The controller 51 may also compare the sensed compression force to predefined values and provide feedback as to whether such sensed compression force is above or below the predefined values.

Although the dynamometer 1 of the present application is described above in the context of measuring force exerted by a hand H, the dynamometer 1 of the present application may just as well be used for measuring force exerted by a person's foot. The disclosure herein is fully applicable to such an embodiment in which force exerted by a person's foot is measured.

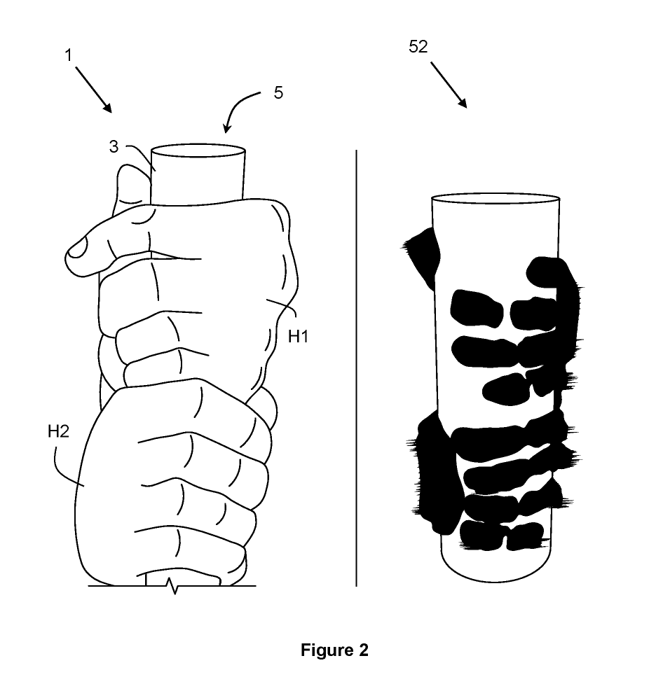

FIG. 2 illustrates on the left a perspective view of the exemplary dynamometer 1, in this embodiment gripped by two hands H1, H2 of the user. FIG. 2 illustrates on the left a prompting device 52, in this case a display on a screen of a computer monitor that displays the sensed compression or contraction forced the person's hands H1, H2 applied to the cylinder 5. The display is in the form of a “weather map” in which darker colors indicate higher force while lighter colors indicate lower force. Unlike prior art dynamometers that were limited to sensing grip force of one hand at a time, the dynamometer 1 of this embodiment may sense grip force of portions of both hands H1, H2 at the same time. This may potentially reduce testing time in half, a significant improvement over the prior art. This feature may also make comparison of strength of left versus right hand easier.

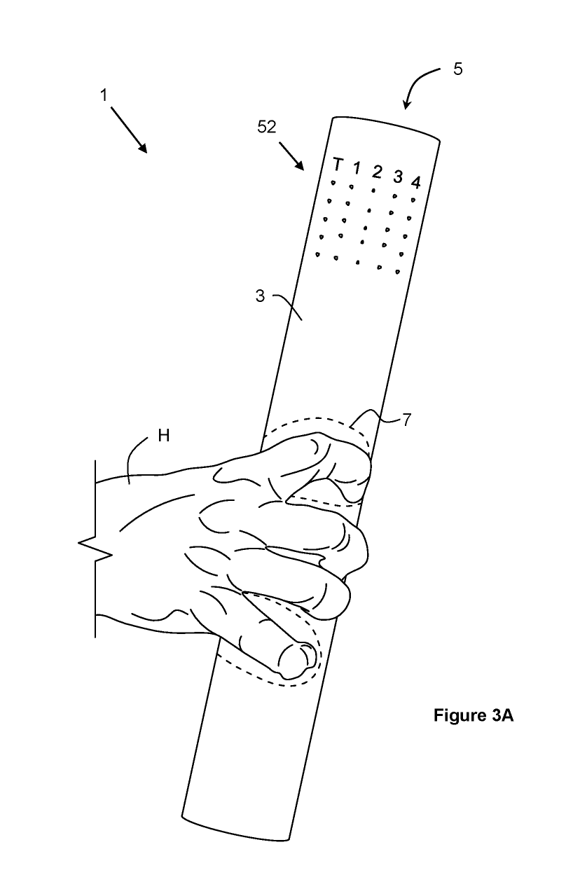

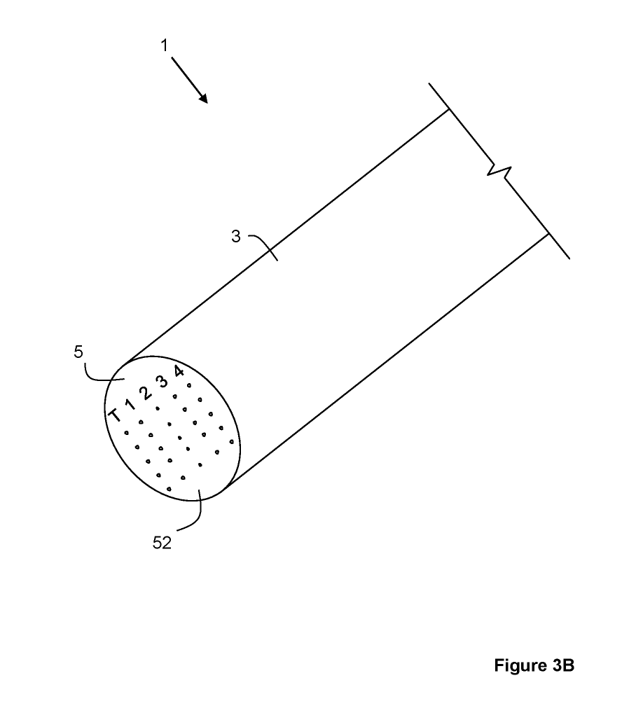

FIG. 3A illustrates a perspective view of an exemplary dynamometer 1. In the embodiment of FIG. 3A, the dynamometer 1 has the outline 7 of a hand drawn on the pad 3 or the cylinder 5 to show hand/finger placement during testing. A user would know where to place her hand H or how to grab the cylinder 5 based on the drawn outline 7 which resembles a hand. In one embodiment, the dynamometer 1 has the outline of a foot drawn on the pad 3 or the cylinder 5 to show foot/toe placement during testing. A user would know where to place her foot based on the drawn outline which resembles a foot. In the embodiment of FIG. 3A, the dynamometer 1 may also include a localized prompting device 52 in the form of five columns of five LED located towards the top end of the dynamometer 1. The columns are labeled T (thumb), 1, 2, 3, and 4 indicating the finger with which each column is associated. The five LED within a column light up in a “fuel gauge” fashion in which a higher number of LED indicates higher sensed force while a lower number of LED indicates lower sensed force for the corresponding finger.

FIG. 3B illustrates a perspective view of another exemplary dynamometer 1. In the embodiment of FIG. 3B, the dynamometer 1 is similar to that of FIG. 3A except that the localized prompting device 52 in the form of five columns of five LED is located at the end of the cylinder 5. The columns are again labeled T (thumb), 1, 2, 3, and 4 indicating the finger with which each column is associated. The five LED within a column light up in a “fuel gauge” fashion in which a higher number of LED indicates higher force while a lower number of LED indicates lower force for the corresponding finger.

The prompting device 52, remote or local, may also include an audio device (e.g., a speaker) in which a louder sound indicates higher force while a lower sound indicates lower force, for example.

The prompting device 52 may also indicate the sensed exerted force as a single number (e.g., pounds, pounds per square inch, etc.) for the whole hand or for individual portions (e.g., fingers F1-F4) of the hand. For example, the controller 51 may aggregate the information received from the distributed sensor 3 to calculate an equivalent location of the sensed compression force throughout the surface area by locating a center of mass of the exerted force over the surface area of the pad 3. A center of mass is a defined term and may mean an arithmetic mean of all points weighted by a local density or specific weight. The controller 51 may determine the center of mass for the whole pad 3 or local centers of mass corresponding to the individual fingers F1-F4 and thumb T. With that information, the controller 51 may then output the measurement number (e.g., pounds, pounds per square inch, etc.) corresponding to the centers of mass.

In the example of FIG. 1, the controller 51 may output a single number (e.g., pounds, pounds per square inch, etc.) by aggregating the forced sensed for the whole pad 3. But notice again that, in the example of FIG. 1, almost all of the force is being applied by the tip of the fingers F1-F4 and therefore five (four of them visible in FIG. 1 and the thumb that is not visible) force centers are clearly discernible on the “weather map.” The controller 51 may output five numbers each corresponding to one of the force centers and, thus, a number per finger or thumb.

The controller 51 may provide the feedback information to the prompting device 52 and, instead or in addition, store the information in memory 58 or drive 59 for later use or provide the feedback information to a communications network (e.g., Internet) via I/O Ports 54 or wireless interfaces 56. This information may then be sent remotely and studied/analyzed by medical personnel such as doctors or therapists. By reading the results from a computer, the doctor or therapist may easily monitor a patient's performance and does not need to notate the results because the data may automatically be stored in the computer. Such a system will keep accurate records and save time.

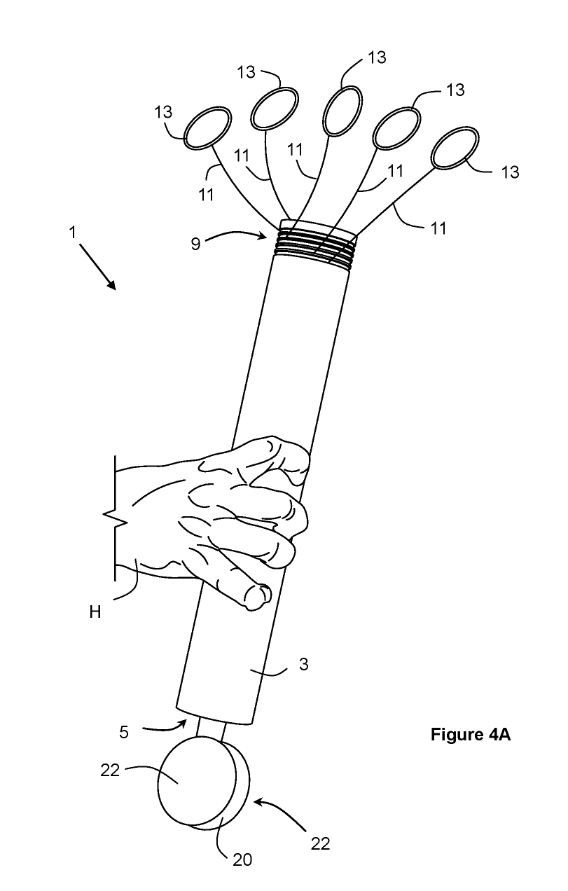

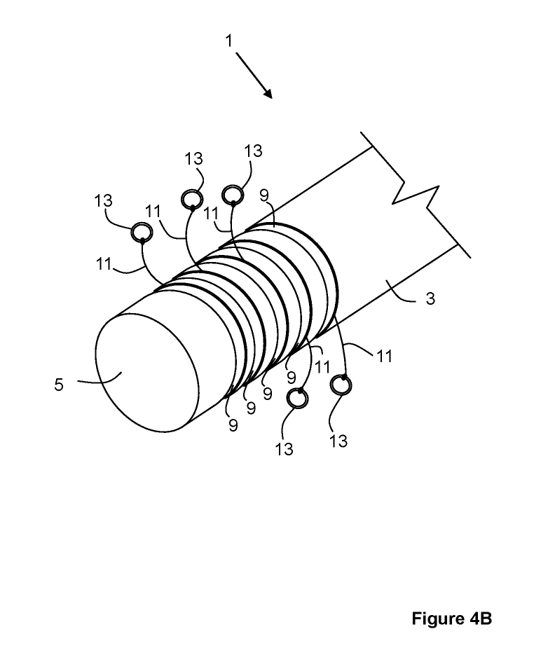

FIGS. 4A and 4B illustrate perspective and magnified views of another exemplary dynamometer 1. In the illustrated embodiment, the dynamometer's cylinder 5 has five grooves 9 on the pad 3 near the top of the cylinder 5. Each groove 9 is associated with a finger. Five strings 11 have one end wrapped around the grooves 9, one string 11 per groove 9. Each string 11 has a band 13 (e.g., ring, square, etc.) attached to the opposite end. This embodiment is capable of measuring expansion or spreading force of the hand's fingers F1-F4 and thumb T. The user would insert her fingers F1-F4 and thumb T in the bands 13 and open her hand. The expansion force of the fingers F1-F4 and thumb T would cause the string or strings 11 to squeeze the distributed sensor 3. Since each groove 9 is associated with a finger, the measured force or pressure for the groove 9 would give an indication as to the expansion or spreading force of that finger and, cumulatively, the hand. Similar to the embodiments above, a prompting device 52 (e.g., whether map, fuel gauge) may be used to display feedback as to the force exerted by each finger or the total hand expansion force. Although in the illustrated embodiment five sets of strings 11 and bands 13 are shown, more or less than five sets of strings 11 and bands 13 may be used.

FIG. 4A further illustrates a pinching dynamometer accessory 20 located attached to the bottom of the dynamometer 1. The pinching dynamometer accessory 20 may include two pads 22 disposed back-to-back. Each pad 22 has a distributed sensor operatively connected to the controller 51 and the prompting device 52 as described above. A user would use a finger and thumb to pinch the pinching dynamometer accessory 20. Since the accessory 20 includes back-to-back distributed sensors 22, the dynamometer may measure pinching force applied based on the force sensed by each sensor 22. The prompting device 52 may display the sensed pinching force applied to the accessory 20. Similar to the embodiments above, the prompting device 52 (e.g., whether map, fuel gauge) may be used to display feedback as to the force exerted by each finger or the total pinching force.

As used herein, an “operable connection” or “operable coupling,” or a connection by which entities are “operably connected” or “operably coupled” is one in which the entities are connected in such a way that the entities may perform as intended. An operable connection may be a direct connection or an indirect connection in which an intermediate entity or entities cooperate or otherwise are part of the connection or are in between the operably connected entities. In the context of signals, an “operable connection,” or a connection by which entities are “operably connected,” is one in which signals, physical communications, or logical communications may be sent or received. Typically, an operable connection includes a physical interface, an electrical interface, or a data interface, but it is to be noted that an operable connection may include differing combinations of these or other types of connections sufficient to allow operable control. For example, two entities can be operably connected by being able to communicate signals to each other directly or through one or more intermediate entities like a processor, operating system, a logic, software, or other entity. Logical or physical communication channels can be used to create an operable connection.

While example systems, methods, and so on, have been illustrated by describing examples, and while the examples have been described in considerable detail, it is not the intention to restrict or in any way limit the scope of the appended claims to such detail. It is, of course, not possible to describe every conceivable combination of components or methodologies for purposes of describing the systems, methods, and so on, described herein. Additional advantages and modifications will readily appear to those skilled in the art. Therefore, the invention is not limited to the specific details, and illustrative examples shown or described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (3D. Ed. 1995).

1. A dynamometer comprising:
a sensor or plurality of sensors distributed within a pad or fabric, the pad or fabric disposed for gripping by at least a portion of a human hand or foot such that grip strength of particular elements of the human hand or foot is sensed by the sensor or plurality of sensors distributed within a pad or fabric.

2. The dynamometer of claim 1, wherein the pad or fabric has at least 91 cm² surface area.

3. The dynamometer of claim 1, comprising:
a controller configured to receive signals from the sensor or plurality of sensors distributed within the pad or fabric corresponding to the sensed grip strength, and
a prompting device operably connected to the controller and configured to produce visual or audio signals corresponding to the sensed grip strength.

4. The dynamometer of claim 1, comprising:
a controller configured to receive signals from the sensor or plurality of sensors distributed within the pad or fabric corresponding to the sensed grip strength, and
a prompting device operably connected to the controller and configured to produce one or more signals corresponding to the sensed grip strength, the one or more signals corresponding to respective one or more fingers or thumb of the human hand or one or more toes of the foot.

5. The dynamometer of claim 1, wherein the pad or fabric has an outline or representation of a hand thereon to indicate hand and finger placement for the gripping by the human hand or the pad or fabric has an outline or representation of a foot thereon to indicate foot and toe placement for the gripping by the human foot.

6. The dynamometer of claim 1, comprising:
a controller configured to receive signals from the sensor or plurality of sensors distributed within the pad or fabric corresponding to the sensed grip strength and to produce feedback signals corresponding to a map of the grip strength sensed by the sensor or plurality of sensors; and
a prompting device operably connected to the controller and configured to display the map of the grip strength sensed by the sensor or plurality of sensors.

7. The dynamometer of claim 1, comprising:
a controller configured to receive signals from the sensor or plurality of sensors distributed within the pad or fabric corresponding to the sensed grip strength and to produce feedback signals corresponding to a map of the grip strength sensed by the sensor or plurality of sensors; and
a prompting device operably connected to the controller and configured to display the map of the grip strength sensed by the sensor or plurality of sensors, wherein the map shows areas of the pad or fabric where sensed grip strength is relative high in a first color and areas of the pad or fabric where sensed grip strength is relative low in a second color different from the first color.

8. A dynamometer comprising:
a sensor embedded within and distributed uniformly within a pad of at least 91 cm² surface area, the pad wrapped and configured for gripping by a human hand such that grip force of particular elements of the human hand is sensed by the sensor.

9. The dynamometer of claim 8, comprising:
a controller configured to receive signals from the sensor, and
a prompting device operably connected to the controller and configured to produce visual or audio signals corresponding to the grip force of the particular elements of the human hand.

10. The dynamometer of claim 8, comprising:
a controller configured to receive signals from the sensor, and
a prompting device operably connected to the controller and configured to produce five signals corresponding to the grip force, four of the five signals corresponding to respective fingers of the human hand and a fifth signal of the five signals corresponding to a thumb of the human hand.

11. The dynamometer of claim 8, wherein the pad has an outline of a hand thereon to indicate hand and finger placement for the gripping by the human hand.

12. The dynamometer of claim 8, comprising:
a controller configured to receive signals from the sensor and to produce feedback signals corresponding to a map of the grip force sensed by the sensor; and
a prompting device operably connected to the controller and configured to display the map of the grip force sensed by the sensor.

13. The dynamometer of claim 8, comprising:
a controller configured to receive signals from the sensor and to produce feedback signals corresponding to a map of the grip force sensed by the sensor; and
a prompting device operably connected to the controller and configured to display the map of the grip force sensed by the sensor, wherein the map shows areas of the pad of high sensed grip force in a different color from areas of the pad of low sensed grip force.

14. The dynamometer of claim 8, comprising:
a string and a band, the band operably attached to the string, the string operably attached to the pad such that force exerted on the band is sensed by the sensor.

15. A dynamometer comprising:
a cylinder;
a pad having embedded within a sensor distributed uniformly within the pad, the pad wrapped around the cylinder,
the combination of the cylinder and pad configured for gripping by a human hand such that compression force of particular elements of the human hand is sensed by the sensor.

16. The dynamometer of claim 15, wherein the pad has at least 91 cm² surface area.

17. The dynamometer of claim 15, comprising:
a controller configured to receive signals from the sensor, and
a prompting device operably connected to the controller and configured to produce visual or audio signals corresponding to the compression force.

18. The dynamometer of claim 15, comprising:
a controller configured to receive signals from the sensor, and
a prompting device operably connected to the controller and configured to produce five signals corresponding to the compression force, four of the five signals corresponding to respective fingers of the human hand and a fifth signal of the five signals corresponding to a thumb of the human hand,
wherein the pad has an outline of a hand thereon to indicate hand and finger placement for the gripping by the human hand.

19. The dynamometer of claim 8, comprising:
a controller configured to receive signals from the sensor and to produce feedback signals corresponding to a map of the compression force sensed by the sensor; and
a prompting device operably connected to the controller and configured to display the map of the compression force sensed by the sensor, wherein the map shows areas of the pad of high sensed compression force in a different color from areas of the pad of low sensed compression force.

20. The dynamometer of claim 15, comprising:
a string and a band, the band operably attached to the string, the string operably attached to the pad such that force exerted on the band is sensed by the sensor.
